(19) United States
(12) Patent Application Publication
(10) Pub. No.: US 2024/0310298 A1
(43) Pub. Date: Sep. 19, 2024

(54) METHOD OF ANALYZING TWO ANALYTES WITHIN A PRESCRIBED TIME PERIOD FOR MEDICAL PURPOSES USING AN ENHANCED NONINVASIVE RF ANALYTE DETECTION DEVICE

(71) Applicant: KNOW LABS, INC., Seattle, WA (US)

(72) Inventor: JOHN CRONIN, Seattle, WA (US)

(21) Appl. No.: 18/606,921

(22) Filed: Mar. 15, 2024

Related U.S. Application Data
(60) Provisional application No. 63/490,913, filed on Mar. 17, 2023.

(51) Int. Cl. G01N 22/00; G16C 20/20; G16C 20/64; G16H 50/30

(52) U.S. Cl. CPC G01N 22/00; G16C 20/20; G16C 20/64; G16H 50/30

(57) ABSTRACT

A method includes measuring multiple analytes using a noninvasive RF analyte detection device in which a program module is used to program the device, an analyte selection module is used to select the analytes that will be measured, a transmission module that measures the two analytes within a predetermined time period and an integration module that determines a notification to be sent to the user and a notification module that informs the user of the notification and data regarding the two analytes.

### FIELD

The present disclosure is generally related to a noninvasive rf analyte detection device for analysis of a plurality of analytes.

### BACKGROUND

Currently, it is difficult to measure multiple analytes using the same device without extracting blood from a patient. Also, invasive procedures to collect analyte data require multiple tests and processes to determine analyte levels to draw conclusions about the analyte data. Lastly, invasive procedures take time to get results to inform the patient of potential conditions or diseases that would benefit from a quicker, less invasive procedure. Thus, there is a need in the prior art to provide a non-invasive analyte detection device for a plurality of analytes.

### SUMMARY

A non-invasive analyte detection device and method for a plurality of analytes. A method described herein includes measuring multiple analytes using a noninvasive RF analyte detection device in which a program module is used to program the device, an analyte selection module is used to select the analytes that will be measured, a transmission module measures the two analytes within a predetermined time period and an integration module determines a notification to be sent to the user and a notification module informs the user of the notification and data regarding the two analytes.

A method to measure multiple analytes using a noninvasive RF analyte detection device can include providing a noninvasive RF analyte detection device; executing an analyte selection module to select a plurality of analytes to measure; executing an integration module; and based upon the control of the integration module, measuring each of the plurality of analytes over a prescribed period of time using the noninvasive RF analyte detection device, wherein the prescribed period of time is determined based on the plurality of analytes being measured; and executing a notification module based on the measurement of the plurality of analytes.

A non-invasive analyte detection system described herein can include a non-invasive radio-frequency (RF) analyte detection device that includes one or more transmit antennas configured to transmit RF analyte detection signals from the one or more transmit antennas into a user, and one or more receive antennas that receive return RF analyte signals that result from the RF analyte detection signals transmitted into the user; the non-invasive RF analyte detection device is configured to perform an analyte detection routine using the one or more transmit antennas and the one or more receive antennas. The system can also include an analog-to-digital converter connected to the one or more receive antennas, and a database in communication with the non-invasive RF analyte detection device and containing data used to control operation of the non-invasive RF analyte detection device during the analyte detection routine. The database contains a plurality of analyte monitoring data entries that differ from one another, each analyte monitoring data entry includes a reason for conducting the analyte detection routine, a type of disease condition being monitored, a first specific analyte, and a second specific analyte.

Another non-invasive analyte detection system described herein can include a database that is in communication with a non-invasive RF analyte detection device and containing data used to control operation of the non-invasive RF analyte detection device during an analyte detection routine. The database contains a plurality of analyte monitoring data entries that differ from one another, each analyte monitoring data entry includes a reason for conducting the analyte detection routine, a type of disease condition being monitored, a first specific analyte, and a second specific analyte.

An analyte monitoring method described herein can include receiving electronic inputs from a user that include an entered reason for conducting the analyte monitoring and an entered type of disease condition to be monitored. Based on the received electronic inputs, electronic analyte monitoring data is obtained from an electronic database that contains a plurality of analyte monitoring data entries that differ from one another, each analyte monitoring data entry includes a reason for conducting the analyte monitoring, a type of disease condition, a first specific analyte, and a second specific analyte. The electronic database is in communication with a non-invasive radio-frequency (RF) analyte detection device so that the obtained electronic analyte monitoring data is able to control the non-invasive radio-frequency (RF) analyte detection device to perform an analyte detection routine.

The analyte monitoring method may also include using the obtained electronic analyte monitoring data to control the non-invasive RF analyte detection device to perform the analyte detection routine.

The analyte monitoring method may also include where the obtained electronic analyte includes a frequency of a first RF analyte detection signal associated with the first analyte, and a frequency of a second RF analyte detection signal associated with the second analyte, wherein each analyte monitoring data entry further includes the frequency of the first RF analyte detection signal associated with the first analyte, and the frequency of the second RF analyte detection signal associated with the second analyte.

The analyte monitoring method may also include where the obtained electronic analyte includes a frequency of a return RF analyte signal associated with the first RF analyte detection signal and a frequency of a return RF analyte signal associated with the second RF analyte detection signal, wherein each analyte monitoring data entry further includes the frequency of the return RF analyte signal associated with the first RF analyte detection signal and the frequency of the return RF analyte signal associated with the second RF analyte detection signal.

The analyte monitoring method may also include where the obtained electronic analyte includes a time at which the first RF analyte detection signal is to be sent, and a time at which the second RF analyte detection signal is to be sent, wherein each analyte monitoring data entry further includes the time at which the first RF analyte detection signal is to be sent, and the time at which the second RF analyte detection signal is to be sent.

The analyte monitoring method may also include where the obtained electronic analyte includes a duration that the first RF analyte detection signal should last, and a duration that the second RF analyte detection signal should last, wherein each analyte monitoring data entry further includes the duration that the first RF analyte detection signal should last, and the duration that the second RF analyte detection signal should last.

The analyte monitoring method may also include storing data obtained by the non-invasive RF analyte detection device during the analyte detection routine in a readings database; and comparing data stored in the readings database with data from a rules database, where the rules database contains a plurality of rule data entries that differ from one another; each rule data entry includes the reason for conducting the analyte monitoring, the type of disease condition, the first specific analyte, a range for the first specific analyte, the second specific analyte, and a range for the second specific analyte. Each rule data entry may further include a notification.

### DRAWINGS

FIG. 1: Illustrates a noninvasive RF analyte detection device, according to an embodiment.

FIG. 2: Illustrates a method of operation of a Base Module, according to an embodiment.

FIG. 3: Illustrates a method of operation of a Program Module, according to an embodiment.

FIG. 4: Illustrates a method of operation of an Analyte Selection Module, according to an embodiment.

FIG. 5: Illustrates a method of operation of a Transmission Module, according to an embodiment.

FIG. 6: Illustrates a method of operation of an Integration Module, according to an embodiment.

FIG. 7: Illustrates a method of operation of a Diagnosis Module, according to an embodiment.

FIG. 8: Illustrates a method of operation of a Monitoring Module, according to an embodiment.

FIG. 9: Illustrates a method of operation of a Screening Module, according to an embodiment.

FIG. 10: Illustrates a method of operation of a Status Module, according to an embodiment.

FIG. 11: Illustrates a method of operation of an Evaluation Module, according to an embodiment.

FIG. 12: Illustrates a method of operation of a Notification Module, according to an embodiment.

FIG. 13: Illustrates an example of a Working Database according to an embodiment.

FIG. 14: Illustrates an example of an Analyte Database according to an embodiment.

FIG. 15: Illustrates an example of a Readings Database according to an embodiment.

FIG. 16: Illustrates an example of a Rules Database according to an embodiment.

### DETAILED DESCRIPTION

Embodiments of the present disclosure will be described more fully hereinafter with reference to the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples.

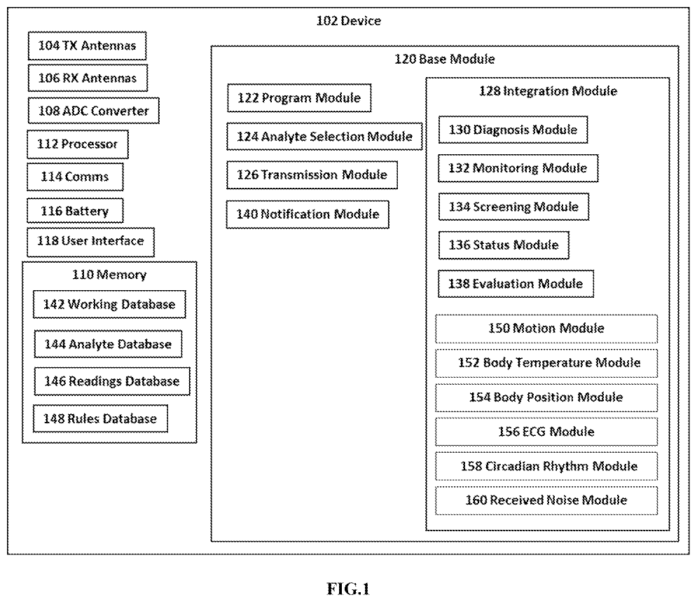

FIG. 1 illustrates a noninvasive RF analyte detection device. The device 102, such as a noninvasive RF analyte detection device, may contain one or more TX antennas 104, one or more RX antennas 106, an ADC converter 108, memory 110, processor 112, communication module 114, battery 116, user interface 118, base module 120 which initiates a program module 122, analyte selection module 124, transmission module 126, integration module 128 and notification module 140. Further, embodiments may include one or more TX antennas 104 which may be integrated into the circuitry arrangement. The one or more TX antennas 104 may be configured to transmit the Activated RF range signals at a pre-defined frequency. The Activated RF range includes signals in a frequency range from 500 MHz to 300 GHz. In one embodiment, the pre-defined frequency may correspond to a range suitable for the human body. For example, at least one of the one or more TX antennas 104 can transmit signals within the Activated RF range, for example at a range of 120-126 GHZ.

At least one of the one or more RX antennas 106 may be configured to receive a response to the transmitted Activated RF range signals. In one embodiment, the Activated RF range signals may be transmitted to the user's skin, and generate a response of electromagnetic energy from parts such as fibrous tissue, muscle, tendons, bones, and the skin. It can be noted that effective monitoring of the blood glucose level can be facilitated by an electromagnetic response of blood molecules, such as pancreatic endocrine hormones, to the transmitted Activated RF range signals. Pancreatic endocrine hormones such as insulin and glucagon are responsible for maintaining sugar or glucose level. Further, the electromagnetic energy responded from the blood molecules may be received by the one or more RX antennas 106.

Further, embodiments may include an ADC Converter 108 which may be coupled to the one or more RX antennas 106. The one or more RX antennas 106 may be configured to receive the responded Activated RF range signals. The ADC 108 may be configured to convert the Activated RF range signals from an analog signal into a digital processor readable format.

Further, embodiments may include a memory 110 may be configured to store the transmitted Activated RF range signals by the one or more TX antennas 104 and receive a responded portion of the transmitted Activated RF range signals from the one or more RX antennas 106. Further, the memory 110 may also store the converted digital processor readable format by the ADC 108. In one embodiment, the memory 110 may include suitable logic, circuitry, and/or interfaces that may be configured to store a machine code and/or a computer program with at least one code section executable by the processor 112. Examples of implementation of the memory 110 may include, but are not limited to, Random Access Memory (RAM), Read Only Memory (ROM), Hard Disk Drive (HDD), and/or a Secure Digital (SD) card.

Further, embodiments may include a processor 112 which may facilitate the operation of the device 102 with the device network to perform functions according to the instructions stored in the memory 110. In one embodiment, the processor 112 may include suitable logic, circuitry, interfaces, and/or code that may be configured to execute a set of instructions stored in the memory 110. The processor 112 may be configured to run the instructions obtained by the device base module 118 to perform polling. The processor 112 may be further configured to collect real-time signals from the one or more TX antennas 104 and the one or more RX antennas 106 and may store the real-time signals in the memory 110. In one embodiment, the real-time signals may be assigned as initial and updated radio frequency (RF) signals. Examples of the processor 112 may be an X86-based processor, a Reduced Instruction Set Computing (RISC) processor, an Application-Specific Integrated Circuit (ASIC) processor, a Complex Instruction Set Computing (CISC) processor, and/or other processors. The processor 112 may be a multicore microcontroller specifically designed to carry multiple operations based upon pre-defined algorithm patterns to achieve the desired result. Further, the processor 112 may take inputs from the device 102 and retain control by sending signals to different parts of the device 102. The processor 112 may consist of a Random Access Memory (RAM) that is used to store data and other results created when the processor 112 is at work. It can be noted that the data is stored temporarily for further processing, such as filtering, correlation, correction, and adjustment. Moreover, the processor 112 carries out special tasks as programs that are pre-stored in the Read Only Memory (ROM). It can be noted that the special tasks carried out by the processor 112 indicate and apply certain actions which trigger specific responses.

Further, the communication module 114 of the device 102 may communicate with the device network via a cloud network. Examples of the communication module 114 may include, but are not limited to, the Internet, a cloud network, a Wireless Fidelity (Wi-Fi) network, a Wireless Local Area Network (WLAN), a Local Area Network (LAN), a telephone line (POTS), Long Term Evolution (LTE), and/or a Metropolitan Area Network (MAN). In one embodiment, various devices may be configured to have a communication module 114 integrated over circuitry arrangement to connect with a device network via various wired and wireless communication protocols, such as the cloud network. Examples of such wired and wireless communication protocols may include, but are not limited to, Transmission Control Protocol and Internet Protocol (TCP/IP), User Datagram Protocol (UDP), Hypertext Transfer Protocol (HTTP), File Transfer Protocol (FTP), Zigbee, EDGE, infrared (IR), IEEE® 802.11, 802.16, cellular communication protocols, and/or Bluetooth® (BT) communication protocols.

Further, embodiments may include a battery 116 which may be disposed over the substrate to power hardware modules of the device 102. The device 102 may be configured with a charging port to recharge the battery 116. It can be noted that the charging of the battery 116 may be wired or wireless means. In one embodiment, the battery 116 may include different models of a lithium-ion battery, such as CR1216, CR2016, CR2032, CR2025, CR2430, CR1220, CR1620, CR1616.

Further, embodiments may include a user interface 118 which may either accept inputs from users or provide outputs to the users or may perform both the actions. For example, the user may input the desired analyte to be detected by the device 102. In one case, a user can interact with the interface(s) using one or more user-interactive objects and devices. The user-interactive objects and devices may comprise user input buttons, switches, knobs, levers, keys, trackballs, touchpads, cameras, microphones, motion sensors, heat sensors, inertial sensors, touch sensors, or a combination of the above. Further, the interface(s) may either be implemented as a Command Line Interface (CLI), a Graphical User Interface (GUI), a voice interface, or a web-based user-interface.

Further, embodiments may include a base module 120 which initiates the program module 122, analyte selection module 124, transmission module 126, integration module 128 and notification module 140 to receive the user inputs, collects the necessary data from the analyte database 144, sends, receives, and stores analyte levels or readings in the readings database 146, and compare the analyte levels to the rules database using the appropriate module determined by the user inputs. In some embodiments, the base module 120 may initiate one or more optional modules from among the motion module 150, the body temperature module 152, the body position module 154, the ECG module 156, the circadian rhythm module 158, and/or the received noise module 160 to determine if the RF data being interpreted by the other modules can be relied upon.

Further, embodiments may include a program module 122 which begins by being initiated by the base module 120. The program module 122 continuously polls to receive the user inputs from the user interface 118. The program module 122 receives the user inputs from the user interface 118. For example, the program module 122 receives user inputs from the user interface 118, such as the reason or purpose of the device 102 and the type of condition the device should monitor or track. For example, the user inputs may define the purpose of the device 102. The purpose of the device can be to assess a condition. The condition can be any suitable condition of a subject in which device 102 measures analytes, such as diagnosis of a disease, monitoring a treatment, for example to evaluate the efficacy or progress of the treatment, screening, monitoring health status, evaluating changes over time, etc. The analytes that are to be detected can be related to the purpose. The program module 122 stores the user inputs in the working database 142. For example, the program module 122 stores the user inputs in the working database including the reason, such as diagnosis a disease, monitor a treatment, screening, monitor health status, evaluate changes over time, etc., the type of condition to assess, such as monitoring or tracking diabetes, high cholesterol, anemia, dehydration, etc. The program module 122 returns to the base module 120.

Further, embodiments may include an analyte selection module 124 which begins by being initiated by the base module 120. The analyte selection module 124 extracts the user inputs from the working database 142. The analyte selection module 124 filters the analyte database 144 on the extracted user inputs. The analyte selection module 124 extracts the data from the analyte database 144. The analyte selection module 124 stores the extracted data from the analyte database 144 in the working database 142. The analyte selection module 124 returns to the base module 120.

Further, embodiments may include a transmission module 126 which begins by being initiated by the base module 120. The transmission module 126 extracts the first analyte monitoring time from the working database 142. The transmission module 126 determines if the current time matches the extracted time to monitor the first analyte from the working database 142. If it is determined that the current time matches the extracted time to monitor the first analyte from the working database 142 the transmission module 126 extracts the first analyte RF transmit signal from the working database 142. The time to monitor the first analyte can be a prescribed period of time, the prescribed period of time based on the condition to be determine based at least in part of measurement of the first analyte. The transmission module 126 sends the RF transmit signal to the TX antenna 104. The transmission module 126 receives the RF signal from the RX antenna 106. The transmission module 126 converts to digital using the ADC converter 108. The transmission module 126 stores the RX converted signal data in memory 110. The transmission module 126 correlates the RF signals with ground truth data to determine the analyte data. The transmission module 126 stores the analyte levels in the Readings database 146. If it is determined that the current time does not match the extracted time to monitor the first analyte from the working database 142 the transmission module 126 extracts the second analyte monitoring time from the working database 142. The transmission module 126 determines if the current time matches the extracted time to monitor the second analyte from the working database 142. If it is determined that the current time does not match the extracted monitoring time of the second analyte from the working database 142 the transmission returns to extracting the monitoring time of the first analyte from the working database 142. If it is determined that the current time matches the extracted time to monitor the second analyte from the working database 142 the transmission module 126 extracts the second analyte RF transmit signal from the working database 142. The time to monitor the second analyte can be a prescribed period of time, the prescribed period of time based on the condition to be determine based at least in part of measurement of the second analyte. The transmission module 126 sends the RF transmit signal to the TX antenna 104. The transmission module 126 receives the RF signal from the RX antenna 106. The transmission module 126 converts to digital using the ADC converter 108. The transmission module 126 stores the RX converted signal data in memory 110. The transmission module 126 correlates the RF signals with ground truth data to determine the analyte data. The transmission module 126 stores the analyte levels in the readings database 146. The transmission module 126 returns to the base module 120.

Further, embodiments may include an integration module 128 which begins by being initiated by the base module 120. The integration module 128 extracts the reason from the working database 142. For example, the reason may be to diagnosis a disease, monitor one or more treatment(s), perform screening, monitor health status, evaluate changes over time, etc. The integration module 128 determines if the diagnosis module 130 should be initiated. For example, if the extracted reason stored in the working database 142 was diagnosis then the integration module 128 determines to initiate the diagnosis module 130 and if the extracted reason was not diagnosis then the integration module 128 determines not to initiate the diagnosis module 130. The reason that is extracted determines the module that integration module initiates. For example, if the reason is diagnosis then the integration module 128 will initiate the diagnosis module 130, if the reason is to monitor then the integration module 128 will initiate the monitor module 132, etc. If it is determined that the diagnosis module 130 should be initiated then the integration module 128 initiates the diagnosis module 130. If it is determined that the diagnosis module 130 should not be initiated then the integration module 128 determines if the monitoring module 132 should be initiated. If it is determined that the monitoring module 132 should be initiated then the integration module 128 initiates the monitoring module 132. If it is determined that the monitoring module 132 should not be initiated then the integration module 128 determines if the screening module 134 should be initiated. If it is determined that the screening module 134 should be initiated then the integration module 128 initiates the screening module 134. If it is determined that the screening module 134 should not be initiated then the integration module 128 determines if the status module 136 should be initiated. If it is determined that the status module 136 should be initiated then the integration module 128 initiates the status module 136. If it is determined that the status module 136 should not be initiated then the integration module 128 determines if the evaluation module 138 should be initiated. If it is determined that the evaluation module 138 should be initiated then the integration module 128 initiates the evaluation module 138. If it is determined that the evaluation module 138 should not be initiated then the integration module 128 returns to the base module 120.

Further, embodiments may include a diagnosis module 130 which begins by being initiated by the integration module 128. The diagnosis module 130 filters the rules database 148 on diagnosis. The diagnosis module 130 extracts the data stored in the readings database 146. The diagnosis module 130 compares the extracted data from the readings database 146 to the filtered rules database 148. The diagnosis module 130 extracts the corresponding notification stored in the rules database 148. The diagnosis module 130 sends the extracted corresponding notification stored in the rules database 148 to the notification module 140. The diagnosis module 130 returns to the integration module 128.

Further, embodiments may include a monitoring module 132 which begins by being initiated by the integration module 128. The monitoring module 132 filters the rules database 148 on monitoring. The monitoring module 132 extracts the data stored in the readings database 146. The monitoring module 132 compares the extracted data from the readings database 146 to the filtered rules database 148. The monitoring module 132 extracts the corresponding notification stored in the rules database 148. The monitoring module 132 sends the extracted corresponding notification stored in the rules database 148 to the notification module 140. The monitoring module 132 returns to the integration module 128.

Further, embodiments may include a screening module 134 which begins by being initiated by the integration module 128. The screening module 134 filters the rules database 148 on screening. The screening module 134 extracts the data stored in the readings database 146. The screening module 134 compares the extracted data from the readings database 146 to the filtered rules database 148. The screening module 134 extracts the corresponding notification stored in the rules database 148. The screening module 134 sends the extracted corresponding notification stored in the rules database 148 to the notification module 140. The screening module 134 returns to the integration module 128.

Further, embodiments may include a status module 136 which begins by being initiated by the integration module 128. The status module 136 filters the rules database 148 on status. The status module 136 extracts the data stored in the readings database 146. The status module 136 compares the extracted data from the readings database 146 to the filtered rules database 148. The status module 136 extracts the corresponding notification stored in the rules database 148. The status module 136 sends the extracted corresponding notification stored in the rules database 148 to the notification module 140. The status module 136 returns to the integration module 128.

Further, embodiments may include an evaluation module 138 which begins by being initiated by the integration module 128. The evaluation module 138 filters the rules database 148 on evaluation. The evaluation module 138 extracts the data stored in the readings database 146. The evaluation module 138 compares the extracted data from the readings database 146 to the filtered rules database 148. The evaluation module 138 extracts the corresponding notification stored in the rules database 148. The evaluation module 138 sends the extracted corresponding notification stored in the rules database 148 to the notification module 140. The evaluation module 138 returns to the integration module 128.

Further, embodiments may include a notification module 140 which begins by being initiated by the base module 120. The notification module 140 continuously polls to receive a notification and data. The notification module 140 receives the notification and data. The notification module 140 displays the notification and data on the user interface 118. The notification module 140 returns to the base module 120.

Further, embodiments may include a working database 142 which is created during the process described in the program module 122 and the analyte selection module 124 and contains the data used to monitor the two analytes for the user. The database contains the reason, such as diagnosis of a disease, monitor a treatment, screening, monitor health status, and evaluate changes over, the type such as the type of condition the user desires to monitor such as diabetes, high cholesterol, anemia, dehydration, etc. the first analyte, the transmit signal of the first analyte, the receive signal of the first analyte, the duration the transmit signal of the first analyte should last, the time in which the transmit signal should be sent for the first analyte, the second analyte, the transmit signal of the second analyte, the receive signal of the second analyte, the duration the transmit signal of the second analyte should last, the time in which the transmit signal should be sent for the second analyte, etc. The database may be unique to the user through the user inputs collected in the program module 122 to monitor two analytes for the purpose of diagnosing a disease, monitoring a treatment, screening, monitoring health status, and evaluating changes over. The time in which the analyte data is collected may be stored in a data file, which may include a schedule to collect the data, such as every hour, daily, weekly, monthly, quarterly, yearly, etc. at certain times during the day, week, month, quarter, year, etc. and/or for a certain time period such as for a day, week, month, quarter, year, etc. In some embodiments, the detection of the analyte may be based upon comparing the transmit signals and received signals to ground truth data collected by medical devices. For example, the database may utilize a module that may be configured to execute an AI correlation between the real-time ground truth data and the RX-converted data. In one embodiment, the AI correlation between the real-time ground truth data and the RX-converted data is executed to determine whether the RX-converted data corresponds to the real-time ground truth data. For example, the module may execute the AI correlation between the real-time ground truth data related to the blood glucose level of the patient as 110 mg/dL corresponding to the radio signal of frequency 122 GHZ, and the 8-bit data corresponding to Activated RF range 140-155 GHz. For example, the memory 110 may store the real-time ground truth data and RX converted data. For example, the memory 110 stores the real-time ground truth data related to the blood glucose level of the patient as 110 mg/dL corresponding to the radio signal of frequency 122 GHZ, and the 8-bit data corresponding to Activated RF range 140-155 GHZ.

Further, embodiments may include an analyte database 144 which contains a plurality of analytes and corresponding data to detect the analytes used by the analyte selection module 124 to extract the data corresponding to the inputs from the user. The database contains the reason, such as diagnosis of a disease, monitor a treatment, screening, monitor health status, and evaluate changes over, the type such as the type of condition the user desires to monitor such as diabetes, high cholesterol, anemia, dehydration, etc. the first analyte, the transmit signal of the first analyte, the receive signal of the first analyte, the duration the transmit signal of the first analyte should last, the time in which the transmit signal should be sent for the first analyte, the second analyte, the transmit signal of the second analyte, the receive signal of the second analyte, the duration the transmit signal of the second analyte should last, the time in which the transmit signal should be sent for the second analyte, etc. The time in which the analyte data is collected may be stored in a data file, which may include a schedule to collect the data, such as every hour, daily, weekly, monthly, quarterly, yearly, etc. at certain times during the day, week, month, quarter, year, etc. and/or for a certain time period such as for a day, week, month, quarter, year, etc.

Further, embodiments may include a readings database 146 which contains the analyte levels collected from the transmission module 126. The database contains the date and time the analyte data was collected, the first analyte, the levels or readings of the first analyte, the second analyte, and the levels or readings of the second analyte. In some embodiments, the database may contain the RF transmit signals sent and received by the TX antenna 104 and RX antenna 106, the filters used by the RX antenna 106, the duration the transmitted signals lasted, etc. In some embodiments, the detection of the analyte may be based upon comparing the transmit signals and received signals to ground truth data collected by medical devices. For example, the database may utilize a module that may be configured to execute an AI correlation between the real-time ground truth data and the RX converted data. In one embodiment, the AI correlation between the real-time ground truth data and the RX-converted data is executed to determine whether the RX-converted data corresponds to the real-time ground truth data. For example, the module may execute the AI correlation between the real-time ground truth data related to the blood glucose level of the patient as 110 mg/dL corresponding to the radio signal of frequency 122 GHZ, and the 8-bit data corresponding to Activated RF range 140-155 GHz. For example, the memory 110 may store the real-time ground truth data and RX converted data. For example, the memory 110 stores the real-time ground truth data related to the blood glucose level of the patient as 110 mg/dL corresponding to the radio signal of frequency 122 GHZ, and the 8-bit data corresponding to Activated RF range 140-155 GHz. In some embodiments, the detection of the analyte of be performed by a module that performs convolution matching on the extracted RF transmit signal that was used by the TX antenna 104 and the received waveform from the RX antenna 106 that was converted to a digital processor readable format and the data extracted from a standard waveform database. For example, convolution is a mathematical operation on two functions, f and g, that produces a third function, f*g, that expresses how the shape of one is modified by the other. The term convolution refers to both the result function and to the process of computing it. It is defined as the integral of the product of the two functions after one is reflected about the y-axis and shifted. The choice of which function is reflected and shifted before the integral does not change the integral result. The integral is evaluated for all values of shift, producing the convolution function. The waveforms stored in memory 110 are matched with each of the individual data entries stored in the standard waveform database. First, the input signal can be decomposed into a set of impulses, each of which can be viewed as a scaled and shifted delta function. Second, the output resulting from each impulse is a scaled and shifted version of the impulse response. Third, the overall output signal can be found by adding these scaled and shifted impulse responses. In other words, if a system's impulse response is known, then we can calculate what the output will be for any possible input signal. In some embodiments, the module may also perform cross-correlation and autocorrelation and store the all the results in memory 110.

Further, embodiments may include a rules database 148 which is used in the process described in the diagnosis module 130, monitoring module 132, screening module 134, status module 136, and evaluation module 138 to determine if the user's analyte levels correspond with a notification that should be sent to the user. The notifications may be notifications, alerts, recommendations, suggestions, etc. to inform the user of the results of their analytes that are being monitored or tracked, such as for diagnosing a disease, monitoring a treatment, screening, monitoring health status, evaluating changes over time, etc. The database contains the reason for the user monitoring, the type of condition the user is monitoring, the first analyte, the levels or readings of the first analyte represented in a range, the second analyte, the levels or readings of the second analyte represented in a range, and the corresponding notification. For example, if the user's reason for monitoring is to diagnose a disease and the type of condition is diabetes the first analyte may be blood glucose and the second analyte may be hemoglobin. The notification may be the user is at risk of diabetes if their blood glucose levels are between 80-130 mg/dL and hemoglobin is between 14-20 g/dL. The user's analytes may be monitored over a period of time; this can be a predetermined period of time for monitoring in order to determine a diagnosis, for example, hemoglobin requires to be monitored for an extended period of time such as over two to three months.

FIG. 2 illustrates a method of operation of a base module 120. The process begins with the base module 120 initiating, at step 200, the program module 122. For example, the program module 122 begins by being initiated by the base module 120. The program module 122 continuously polls to receive the user inputs from the user interface 118. The program module 122 receives the user inputs from the user interface 118. The program module 122 stores the user inputs in the working database 142. The program module 122 returns to the base module 120. The base module 120 initiates, at step 202, the analyte selection module 124. For example, the analyte selection module 124 begins by being initiated by the base module 120. The analyte selection module 124 extracts the user inputs from the working database 142. The analyte selection module 124 filters the analyte database 144 on the extracted user inputs. The analyte selection module 124 extracts the data from the analyte database 144. The analyte selection module 124 stores the extracted data from the analyte database 144 in the working database 142. The analyte selection module 124 returns to the base module 120. The base module 120 initiates, at step 204, the transmission module 126. For example, the transmission module 126 begins by being initiated by the base module 120. The transmission module 126 extracts the first analyte monitoring time from the working database 142. The transmission module 126 determines if the current time matches the extracted time to monitor the first analyte from the working database 142. If it is determined that the current time matches the extracted time to monitor the first analyte from the working database 142 the transmission module 126 extracts the first analyte RF transmit signal from the working database 142. The transmission module 126 sends the RF transmit signal to the TX antenna 104. The transmission module 126 receives the RF signal from the RX antenna 106. The transmission module 126 converts to digital using the ADC converter 108. The transmission module 126 stores the RX converted signal data in memory 110. The transmission module 126 correlates the RF signals with ground truth data to determine the analyte data. The transmission module 126 stores the analyte levels in the Readings database 146. If it is determined that the current time does not match the extracted time to monitor the first analyte from the working database 142 the transmission module 126 extracts the second analyte monitoring time from the working database 142. The transmission module 126 determines if the current time matches the extracted time to monitor the second analyte from the working database 142. If it is determined that the current time does not match the extracted monitoring time of the second analyte from the working database 142 the transmission returns to extracting the monitoring time of the first analyte from the working database 142. If it is determined that the current time matches the extracted time to monitor the second analyte from the working database 142 the transmission module 126 extracts the second analyte RF transmit signal from the working database 142. The transmission module 126 sends the RF transmit signal to the TX antenna 104. The transmission module 126 receives the RF signal from the RX antenna 106. The transmission module 126 converts to digital using the ADC converter 108. The transmission module 126 stores the RX converted signal data in memory 110. The transmission module 126 correlates the RF signals with ground truth data to determine the analyte data. The transmission module 126 stores the analyte levels in the Readings database 146. The transmission module 126 returns to the base module 120.

In some embodiments, the base module 120 may utilize, at step 206, a motion module 150 that includes at least one sensor from the group of an accelerometer, a gyroscope, an inertial movement sensor, or other similar sensor. The motion module 150 may have its own processor or utilize the signal processor 112 to calculate the user's movement. Motion from the user will change the blood volume in a given portion of their body and the blood flow rate in their circulatory system. This may cause noise, artifacts, or other errors in the real-time signals received by the RX antennas 106. The motion module 150 may compare the calculated motion to a motion threshold stored in memory 110. For example, the motion threshold could be movement of more than two centimeters in one second. The motion threshold could be near zero to ensure the user is stationary when measuring to ensure the least noise in the RF signal data. When calculated motion levels exceed the motion threshold, the motion module 150 may flag the RF signals collected at the time stamp corresponding to the motion as potentially inaccurate. In some embodiments, the motion module 150 may compare RF signal data to motion data over time to improve the accuracy of the motion threshold. The motion module 150 may alert the user, such as with an audible beep or warning or a text message or alert to a connected mobile device. The alert would signal the user that they are moving too much to get an accurate measurement. The motion module 150 may update the Readings database 146 with the calculated motion of the user that corresponds with the received RF signal data. In this manner, the motion module 150 may be simplified to just collect motion data and allow the base module 120 to determine if the amount of motion calculated exceeds a threshold that would indicate the received RF signal data is too noisy to be relied upon for a SPO2 measurement.

The base module 120 may utilize, at step 206, a body temperature module 152 that includes at least one sensor from the group of a thermometer, a platinum resistance thermometer (PRT), a thermistor, a thermocouple, or another temperature sensor. The body temperature module 152 may have its own processor or utilize the signal processor 112 to calculate the temperature of the user or the user's environment. The user's body temperature, the environmental temperature, and the difference between the two will change the blood volume in a given part of their body and the blood flow rate in their circulatory system. Variations in temperature from the normal body temperature or room temperature may cause noise, artifacts, or other errors in the real-time signals received by the RX antennas 106. The body temperature module 152 may compare the measured temperature to a threshold temperature stored in memory 110. For example, the environmental temperature threshold may be set at zero degrees Celsius because low temperatures can cause a temporary narrowing of blood vessels which may increase the user's blood pressure. When the measured temperature exceeds the threshold, the body temperature module 152 may flag the RF signals collected at the time stamp corresponding to the temperature as potentially being inaccurate. In some embodiments, the body temperature module 152 may compare RF signal data to temperature data over time to improve the accuracy of the temperature threshold. The body temperature module 152 may alert the user, such as with an audible beep or warning or a text message or alert to a connected mobile device. The alert would signal to the user that their body temperature, or the environmental temperature is not conducive to getting an accurate measurement. The body temperature module 152 update the Readings database 146 with the measured user or environmental temperature that corresponds with the received RF signal data. In this manner, the body temperature module 152 may be simplified to just collect temperature data and allow the base module 120 to determine if the temperature measure exceeds a threshold that would indicate the received RF signal data is too noisy to be relied upon for a SPO2 measurement.

The base module 120 may utilize, at step 206, a body position module 154 that includes at least one sensor from the group of an accelerometer, a gyroscope, an inertial movement sensor, or another similar sensor. The body position module 154 may have its own processor or utilize the signal processor 112 to estimate the user's position. The user's body position may change the blood volume in a given part of their body and the blood flow rate in their circulatory system. This may cause noise, artifacts, or other errors in the real-time signals received by the RX antennas 106. The body position module 154 may compare the estimated position to a body position threshold stored in memory 110. For example, the monitoring device 102 may be on the user's wrist, and the body position threshold may be based on the relative position of the user's hand to their heart. When a user's hand is lower than their heart, their blood pressure will increase, with this effect being more pronounced the longer the position is maintained. Conversely, the higher a user holds their arm above their heart, the lower the blood pressure in their hand. The body position threshold may include some minimum amount of time the estimated body position occurs. When the estimated position exceeds the threshold, the body position module 154 may flag the RF signals collected at the time stamp corresponding to the body position as potentially being inaccurate. In some embodiments, the body position module 154 may compare RF signal data to motion data over time to improve the accuracy of the body position threshold. The body position data may also be used to estimate variations in parameters such as blood pressure that corresponds to the body position data to improve the accuracy of the measurements taken when the user is in that position. The body position module 154 may alert the user, such as with an audible beep or warning or a text message or alert to a connected mobile device. The alert would signal to the user that their body position is not conducive to getting an accurate measurement. The body position module 154 may update the Readings database 146 with the estimated body position data that corresponds with the received RF signal data. In this manner, the body position module 154 may be simplified to just collect temperature data and allow the base module 120 to determine if the body position exceeded a threshold that would indicate the received RF signal data is too noisy to be relied upon for a SPO2 measurement.

The base module 120 may utilize, at step 206, an ECG module 156 that includes at least one electrocardiogram sensor. The ECG module 156 may have its own processor or utilize the signal processor 112 to record the electrical signals that correspond with the user's heartbeat. The user's heartbeat will impact blood flow. Measuring the ECG data may allow the received RF data to be associated with peak and minimum cardiac output so as to create a pulse waveform allowing for the estimation of blood volume at a given point in the wave of ECG data. Variations in blood volume may cause noise, artifacts, or other errors in the real-time signals received by the RX antennas 106. The ECG module 156 may compare the measured cardiac data to a threshold stored in memory 110. For example, the threshold may be a pulse above 160 bpm, as the increased blood flow volume may cause too much noise in the received RF signal data to accurately measure the SPO2. When the ECG data exceeds the threshold, the ECG module 156 may flag the RF signals collected at the time stamp corresponding to the ECG data as potentially being inaccurate. In some embodiments, the ECG module 156 may compare RF signal data to ECG data over time to improve the accuracy of the ECG data threshold or to improve the measurement of SPO2 at a given point in the cycle between peak and minimum cardiac output. The ECG module 156 may alert the user, such as with an audible beep or warning or a text message or alert to a connected mobile device. The alert would signal to the user that their heart rate is not conducive to getting an accurate measurement or requires additional medical intervention. The ECG module 156 may update the Readings database 146 with the measured ECG data that corresponds with the received RF signal data. In this manner, the ECG module 156 may be simplified to just collect ECG data and allow the base module 120 to determine if the ECG data exceeded a threshold that would indicate the received RF signal data is too noisy to be relied upon for a SPO2 measurement.

The base module 120 may utilize, at step 206, a circadian rhythm module 158 that includes at least one sensor measuring actigraphy, wrist temperature, light exposure, and heart rate. The circadian rhythm module 158 may have its own processor or utilize the signal processor 112 to calculate the user's circadian health. Blood pressure follows a circadian rhythm in that it increases upon waking in the morning and decreases during sleeping at night. People with poor circadian health will often have higher blood pressure. These variations in blood pressure can cause noise, artifacts, or other errors or inaccuracies in the real-time signals received by the RX antennas 106. The circadian rhythm module 158 may compare the circadian data to a threshold stored in memory 110. For example, the threshold may be less than 6 hours of sleep in the last 24 hours. When the observed circadian health data exceeds the threshold, the circadian rhythm module 158 may flag the RF signals collected at the time stamp corresponding to circadian health as potentially being inaccurate or needing an adjustment to account for the expected increase in the user's blood pressure. In some embodiments, the circadian rhythm module 158 may compare RF signal data to sleep data over time to improve the accuracy of the circadian rhythm thresholds. The circadian rhythm module 158 may alert the user, such as with an audible beep or warning, or a text message or alert to a connected mobile device. The alert would signal to the user that their recent sleep patterns are not conducive to getting an accurate measurement. The circadian rhythm module 158 may update the Readings database 146 with the measured circadian data that corresponds with the received RF signal data. In this manner, the circadian rhythm module 158 may be simplified to just collect circadian rhythm data and allow the base module 120 to determine if the measure exceeded a threshold that would indicate the received RF signal data is too noisy to be relied upon for a SPO2 measurement, or if an alternative transfer function should be used to compensate for the detected circadian health.

The base module 120 may utilize, at step 206, a received noise module 160 that includes at least one sensor measuring background signals such as RF signals, Wi-Fi, and other electromagnetic signals that could interfere with the signals received by the RX antennas 106. The received noise module 160 may have its own processor or utilize the signal processor 112 to calculate the level of background noise being received. Background noise may interfere with or cause noise, artifacts, or other errors or inaccuracies in the real-time signals received by the RX antennas 106. The received noise module 160 may compare the level and type of background noise to a threshold stored in memory 110. The threshold may be in terms of field strength (volts per meter and ampere per meter) or power density (watts per square meter). For example, the threshold may be RF radiation greater than 300 μW/m2. When the background noise data exceeds the threshold, the received noise module 160 may flag the RF signals collected at the time stamp corresponding to background noise levels as potentially being inaccurate. In some embodiments, the received noise module 160 may compare RF signal data to background noise over time to improve the accuracy of the noise thresholds. The received radiation module may alert the user, such as with an audible beep or warning, a text message, or an alert to a connected mobile device. The alert would signal to the user that the current level of background noise is not conducive to getting an accurate measurement. The received noise module 160 may update the Readings database 146 with the background noise data that corresponds with the received RF signal data. In this manner, the received noise module 160 may be simplified to just collect background noise data and allow the base module 120 to determine if the measure exceeded a threshold that would indicate the received RF signal data is too noisy to be relied upon for a SPO2 measurement, or if an alternative transfer function should be used to compensate for the noise.

The base module 120 initiates, at step 208, the integration module 128. For example, the integration module 128 begins by being initiated by the base module 120. The integration module 128 extracts the reason from the working database 142. The integration module 128 determines if the diagnosis module 130 should be initiated. If it is determined that the diagnosis module 130 should be initiated then the integration module 128 initiates the diagnosis module 130. If it is determined that the diagnosis module 130 should not be initiated then the integration module 128 determines if the monitoring module 132 should be initiated. If it is determined that the monitoring module 132 should be initiated then the integration module 128 initiates the monitoring module 132. If it is determined that the monitoring module 132 should not be initiated then the integration module 128 determines if the screening module 134 should be initiated. If it is determined that the screening module 134 should be initiated then the integration module 128 initiates the screening module 134. If it is determined that the screening module 134 should not be initiated then the integration module 128 determines if the status module 136 should be initiated. If it is determined that the status module 136 should be initiated then the integration module 128 initiates the status module 136. If it is determined that the status module 136 should not be initiated then the integration module 128 determines if the evaluation module 138 should be initiated. If it is determined that the evaluation module 138 should be initiated then the integration module 128 initiates the evaluation module 138. If it is determined that the evaluation module 138 should not be initiated then the integration module 128 returns to the base module 120.

The base module 120 initiates, at step 210, the notification module 140. For example, the notification module 140 begins by being initiated by the base module 120. The notification module 140 continuously polls to receive a notification and data. The notification module 140 receives the notification and data. The notification module 140 displays the notification and data on the user interface 118. The notification module 140 returns to the base module 120.

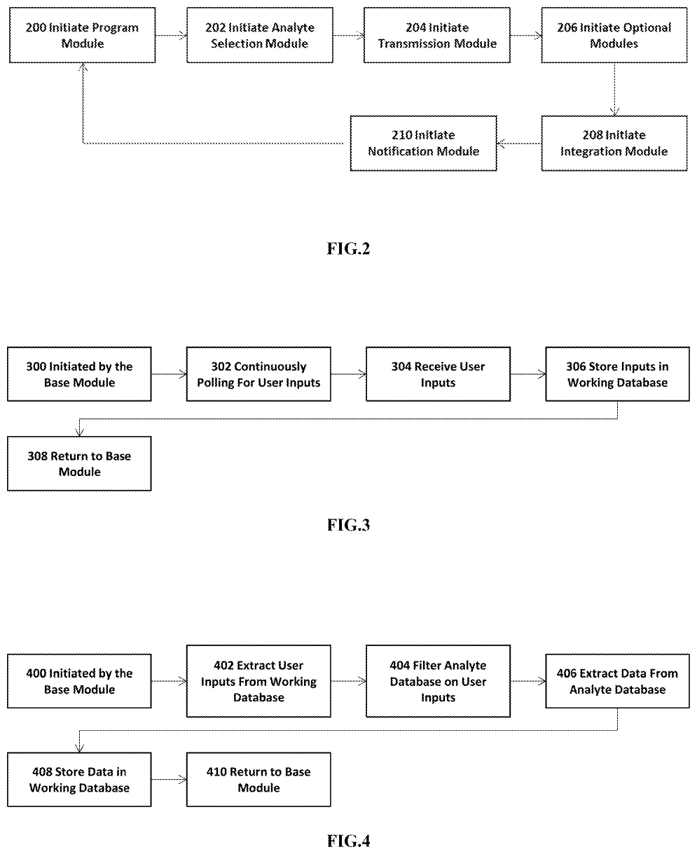

FIG. 3 illustrates a method of operation of a program module 122. The process begins with the program module 122 being initiated, at step 300, by the base module 120. For example, the program module 122 is initiated to collect user input data from the user to determine the purpose of the device 102. In some embodiments, the user inputs may be preprogrammed into the device 102 prior to use. The program module 122 continuously polls, at step 302, to receive the user inputs from the user interface 118. For example, the program module 122 continuously polls to receive user inputs from the user interface 118, such as the reason or purpose of the device 102 and the type of condition the device should monitor or track. For example, the user inputs may define the purpose of the device 102, such as diagnosis a disease, monitor a treatment, screening, monitor health status, evaluate changes over time, etc. Also, the user inputs may allow the device 102 to track or monitor a type of condition that is related to the purpose or reason of the device 102, such as diagnosis diabetes, monitor high cholesterol treatments, screening for anemia, monitor dehydration levels, evaluate hydration over a period of time, etc. The program module 122 receives, at step 304, the user inputs from the user interface 118. For example, the program module 122 receives user inputs from the user interface 118, such as the reason or purpose of the device 102 and the type of condition the device should monitor or track. For example, the user inputs may define the purpose of the device 102, such as diagnosis a disease, monitor a treatment, screening, monitor health status, evaluate changes over time, etc. Also, the user inputs may allow the device 102 to track or monitor a type of condition that is related to the purpose or reason of the device 102, such as diagnosis diabetes, monitor high cholesterol treatments, screening for anemia, monitor dehydration levels, evaluate hydration over a period of time, etc. The program module 122 stores, at step 306, the user inputs in the working database 142. For example, the program module 122 stores the user inputs in the working database including the reason, such as diagnosis a disease, monitor a treatment, screening, monitor health status, evaluate changes over time, etc., the type of condition to monitor or track, such as diabetes, high cholesterol, anemia, dehydration, etc. The program module 122 returns, at step 308, to the base module 120. For example, once the user inputs have been collected and stored the program module 122 returns to the base module 120.

FIG. 4 illustrates a method of operation of an analyte selection module 124. The process begins with the analyte selection module 124 being initiated, at step 400, by the base module 120. For example, the analyte selection module 124 may be initiated once the user inputs are collected and stored by the program module 122. The analyte selection module 124 extracts, at step 402, the user inputs from the working database 142. For example, the analyte selection module extracts the user inputs, such as the reason and type, from the working database. For example, the analyte selection module 124 extracts the reason, such as diagnosis a disease, monitor a treatment, screening, monitor health status, evaluate changes over time, etc., the type of condition to monitor or track, such as diabetes, high cholesterol, anemia, dehydration, etc. The analyte selection module 124 filters, at step 404, the analyte database 144 on the extracted user inputs. For example, the analyte selection module 124 filters the analyte database 144 on the extracted user inputs, such as diagnosis a disease, monitor a treatment, screening, monitor health status, evaluate changes over time, etc., the type of condition to monitor or track, such as diabetes, high cholesterol, anemia, dehydration, etc. The analyte selection module 124 extracts, at step 406, the data from the analyte database 144. For example, the analyte selection module 124 extracts the corresponding data related to the user inputs, such as the reason and type. The data extracted from the analyte database 144 may be the first analyte, the transmit signal of the first analyte, the receive signal of the first analyte, the duration the transmit signal of the first analyte should last, the time in which the transmit signal should be sent for the first analyte, the second analyte, the transmit signal of the second analyte, the receive signal of the second analyte, the duration the transmit signal of the second analyte should last, the time in which the transmit signal should be sent for the second analyte, etc. The time in which the analyte data is collected may be stored in a data file, which may include a schedule to collect the data, such as every hour, daily, weekly, monthly, quarterly, yearly, etc. at certain times during the day, week, month, quarter, year, etc. and/or for a certain time period such as for a day, week, month, quarter, year, etc. The analyte selection module 124 stores, at step 408, the extracted data from the analyte database 144 in the working database 142. For example, the analyte selection module 124 stores the extracted data from the analyte database 144 in the working database 142 including, the first analyte, the transmit signal of the first analyte, the receive signal of the first analyte, the duration the transmit signal of the first analyte should last, the time in which the transmit signal should be sent for the first analyte, the second analyte, the transmit signal of the second analyte, the receive signal of the second analyte, the duration the transmit signal of the second analyte should last, the time in which the transmit signal should be sent for the second analyte, etc. The time in which the analyte data is collected may be stored in a data file, which may include a schedule to collect the data, such as every hour, daily, weekly, monthly, quarterly, yearly, etc. at certain times during the day, week, month, quarter, year, etc. and/or for a certain time period such as for a day, week, month, quarter, year, etc. The analyte selection module 124 returns, at step 410, to the base module 120.

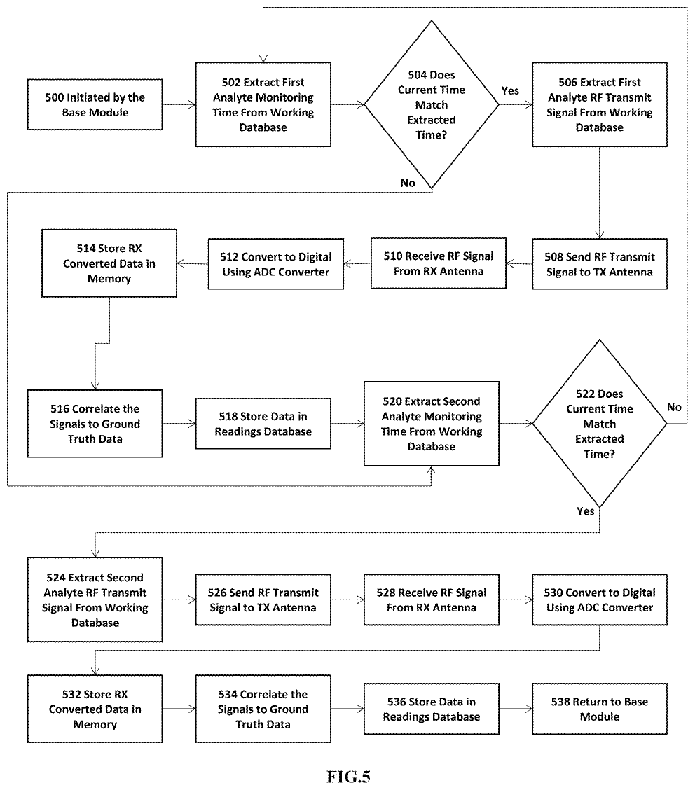

FIG. 5 illustrates a method of operation of a transmission module 126. The process begins with the transmission module 126 being initiated, at step 500, by the base module 120. For example, the base module 120 initiates the transmission module 126 to collect the analyte data using the RF signals stored in the working database 142 and stores the analyte readings or levels in the readings database 146. In some embodiments, the transmission module 126 may send a plurality of RF signals, receive a plurality of RF signals, and determine a plurality of analyte readings or levels. In some embodiments, the transmission module 142 may send, receive, collect, and determine data for an extended period of time such as a day, week, month, quarter, year, etc. as determined by the extracted time to measure the first and second analyte stored in the working database 142. The transmission module 126 extracts, at step 502, the first analyte monitoring time from the working database 142. For example, the transmission module 126 extracts the first analyte monitoring time from the working database 142, such as a schedule to collect the data, such as every hour, daily, weekly, monthly, quarterly, yearly, etc. at certain times during the day, week, month, quarter, year, etc. and/or for a certain time period such as for a day, week, month, quarter, year, etc. In some embodiments, the analyte monitoring time may be stored as a data file.

The transmission module 126 determines, at step 504, if the current time matches the extracted time to monitor the first analyte from the working database 142. For example, the transmission module 126 may compare the days and times stored in the extracted data file to monitor the first analyte to the current date and time and if the current time matches one of the days or times, or both, stored in the data file, the transmission module 126 extracts the RF transmit signal stored in the working database 142. If it is determined that the current time matches the extracted time to monitor the first analyte from the working database 142 the transmission module 126 extracts, at step 506, the first analyte RF transmit signal from the working database 142. For example, the transmission module 126 extracts the RF transmit signal for the first analyte stored in the working database 142. The transmission module 126 sends, at step 508, the RF transmit signal to the TX antenna 104. For example, the one or more TX antennas 104 may be configured to transmit the Activated RF range signals at a pre-defined frequency. In one embodiment, the pre-defined frequency may correspond to a range suitable for the human body. For example, the one or more TX antennas 104 transmit Activated RF range signals at a range of 120-126 GHZ.

The transmission module 126 receives, at step 510, the RF signal from the RX antenna 106. For example, the one or more RX antennas 106 may be configured to receive the responded portion of the Activated RF range signals. In one embodiment, the Activated RF range signals may be transmitted into the user, and electromagnetic energy may be responded from many parts such as fibrous tissue, muscle, tendons, bones, and the skin. The transmission module 126 converts, at step 512, to digital using the ADC converter 108. For example, the ADC 108 may be configured to convert the Activated RF range signals from an analog signal into a digital processor readable format.

The transmission module 126 stores, at step 514, the RX converted signal data in memory 110. For example, the transmission module 126 stores the received signal from the RX antenna 106 that has been converted to a digital processor readable format in memory 110. The transmission module 126 correlates, at step 516, the RF signals with ground truth data to determine the analyte data. For example, the transmission module 126 may be configured to execute an AI correlation between the real-time ground truth data and the RX converted data. In one embodiment, the AI correlation between the real-time ground truth data and the RX-converted data is executed to determine whether the RX-converted data corresponds to the real-time ground truth data. The ground truth data may be determined by a medical device that identifies the analyte levels or number at the time a waveform was transmitted and received. Machine learning processes may be performed to identify specific analyte waveforms, which complex responded signals from stepped frequencies transmit signals, that can be related to analyte levels. The memory 110 is used in real time to compare received waveforms from the RX antenna 106 to a standard waveform database stored in memory 110 to identify the analyte number for the received waveform from the RX antenna 106. In one embodiment, the standard waveform database may be configured to store the filtered RF signal received from the one or more RX antennas 106 of the device 102. The standard waveform database may store the signal waveforms for the TX antenna 104 and the received signal waveforms for the RX antenna 106. The database may include the analyte readings with the corresponding signal waveform, received waveform and the TX antenna 104 and RX antenna 106 that were used.

The transmission module 126 stores, at step 518, the first analyte levels in the readings database 146. For example, the transmission module 126 stores the user's first analyte levels in the readings database 146. If it is determined that the current time does not match the extracted time to monitor the first analyte from the working database 142 the transmission module 126 extracts, at step 520, the second analyte monitoring time from the working database 142. For example, the transmission module 126 extracts the second analyte monitoring time from the working database 142, such as a schedule to collect the data, such as every hour, daily, weekly, monthly, quarterly, yearly, etc. at certain times during the day, week, month, quarter, year, etc. and/or for a certain time period such as for a day, week, month, quarter, year, etc. In some embodiments, the analyte monitoring time may be stored as a data file. The transmission module 126 determines, at step 522, if the current time matches the extracted time to monitor the second analyte from the working database 142. If it is determined that the current time does not match the extracted monitoring time of the second analyte from the working database 142 the transmission returns to extracting the monitoring time of the first analyte from the working database 142. For example, the transmission module 126 may compare the days and times stored in the extracted data file to monitor the second analyte to the current date and time and if the current time matches one of the days or times, or both, stored in the data file, the transmission module 126 extracts the RF transmit signal stored in the working database 142.

If it is determined that the current time matches the extracted time to monitor the second analyte from the working database 142 the transmission module 126 extracts, at step 524, the second analyte RF transmit signal from the working database 142. For example, the transmission module 126 extracts the RF transmit signal for the second analyte stored in the working database 142. The transmission module 126 sends, at step 526, the RF transmit signal to the TX antenna 126. For example, the one or more TX antennas 104 may be configured to transmit the Activated RF range signals at a pre-defined frequency. In one embodiment, the pre-defined frequency may correspond to a range suitable for the human body. For example, the one or more TX antennas 104 transmit Activated RF range signals at a range of 120-126 GHz. The transmission module 126 receives, at step 528, the RF signal from the RX antenna 126. For example, the one or more RX antennas 106 may be configured to receive the responded portion of the Activated RF range signals. In one embodiment, the Activated RF range signals may be transmitted to the user's skin, and electromagnetic energy may be responded from many parts such as fibrous tissue, muscle, tendons, bones, and the skin. The transmission module 126 converts, at step 530, to digital using the ADC converter 108. For example, the ADC 108 may be configured to convert the Activated RF range signals from an analog signal into a digital processor readable format. The transmission module 126 stores, at step 532, the RX converted signal data in memory 110. For example, the transmission module 126 stores the received signal from the RX antenna 106 that has been converted to a digital processor readable format in memory 110.

The transmission module 126 correlates, at step 534, the RF signals with ground truth data to determine the analyte data. For example, the transmission module 126 may be configured to execute an AI correlation between the real-time ground truth data and the RX converted data. In one embodiment, the AI correlation between the real-time ground truth data and the RX-converted data is executed to determine whether the RX-converted data corresponds to the real-time ground truth data. The ground truth data may be determined by a medical device that identifies the analyte levels or number at the time a waveform was transmitted and received. Machine learning processes may be performed to identify specific analyte waveforms, which complex responded signals from stepped frequencies transmit signals, that can be related to analyte levels. The memory 110 is used in real time to compare received waveforms from the RX antenna 106 to a standard waveform database stored in memory 110 to identify the analyte number for the received waveform from the RX antenna 106. In one embodiment, the standard waveform database may be configured to store the filtered RF signal received from the one or more RX antennas 106 of the device 102. The standard waveform database may store the signal waveforms for the TX antenna 104 and the received signal waveforms for the RX antenna 106. The database may include the analyte readings with the corresponding signal waveform, received waveform and the TX antenna 104 and RX antenna 106 that were used.

The transmission module 126 stores, at step 536, the analyte levels in the readings database 146. For example, the transmission module 126 stores the user's second analyte levels in the readings database 146. The transmission module 126 returns, at step 538, to the base module 120. For example, the transmission module 126 returns to the base module 120 once the first and second analyte readings have been stored in the readings database 146. In some embodiments, the transmission module 126 may be continuously executed until the transmission module 126 has collected enough data on each of the two analytes as determined by the monitoring times for the first and second analyte stored in the working database 142 as a data file.

FIG. 6 illustrates a method of operation of the integration module 128. The process begins with the integration module 128 being initiated, at step 600, by the base module 120. For example, the integration module 128 is initiated once the transmission module 126 has collected and stored all the analyte data for the first and second analyte over the necessary time period as determined by the data files stored in the working database 142 to determine when and for how long to collect the analyte data. The integration module 128 extracts, at step 602, the reason from the working database 142. For example, the integration module may extract the reason, such as diagnosis, monitor, screening, status, evaluation, etc. from the working database 142. The reason that is extracted determines the module that integration module initiates. For example, if the reason is diagnosis then the integration module 128 will initiate the diagnosis module 130, if the reason is to monitor then the integration module 128 will initiate the monitor module 132, etc. The integration module 128 determines, at step 604, if the diagnosis module 130 should be initiated. For example, if the extracted reason stored in the working database 142 was diagnosis then the integration module 128 determines to initiate the diagnosis module 130 and if the extracted reason was not diagnosis then the integration module 128 determines not to initiate the diagnosis module 130. If it is determined that the diagnosis module 130 should be initiated then the integration module 128 initiates, at step 606, the diagnosis module 130. For example, the diagnosis module 130 begins by being initiated by the integration module 128. The diagnosis module 130 filters the rules database 148 on diagnosis. The diagnosis module 130 extracts the data stored in the readings database 146. The diagnosis module 130 compares the extracted data from the readings database 146 to the filtered rules database 148. The diagnosis module 130 extracts the corresponding notification stored in the rules database 148. The diagnosis module 130 sends the extracted corresponding notification stored in the rules database 148 to the notification module 140. The diagnosis module 130 returns to the integration module 128.

If it is determined that the diagnosis module 130 should not be initiated then the integration module 128 determines, at step 608, if the monitoring module 132 should be initiated. For example, if the extracted reason stored in the working database 142 was monitor then the integration module 128 determines to initiate the monitor module 132 and if the extracted reason was not monitor then the integration module 128 determines not to initiate the monitor module 132. If it is determined that the monitoring module 132 should be initiated then the integration module 128 initiates, at step 610, the monitoring module 132. For example, the monitoring module 132 begins by being initiated by the integration module 128. The monitoring module 132 filters the rules database 148 on monitoring. The monitoring module 132 extracts the data stored in the readings database 146. The monitoring module 132 compares the extracted data from the readings database 146 to the filtered rules database 148. The monitoring module 132 extracts the corresponding notification stored in the rules database 148. The monitoring module 132 sends the extracted corresponding notification stored in the rules database 148 to the notification module 140. The monitoring module 132 returns to the integration module 128.

If it is determined that the monitoring module 132 should not be initiated then the integration module 128 determines, at step 612, if the screening module 134 should be initiated. For example, if the extracted reason stored in the working database 142 was screening then the integration module 128 determines to initiate the screening module 134 and if the extracted reason was not screening then the integration module 128 determines not to initiate the screening module 134. If it is determined that the screening module 134 should be initiated then the integration module 128 initiates, at step 614, the screening module 134. For example, the screening module 134 begins by being initiated by the integration module 128. The screening module 134 filters the rules database 148 on screening. The screening module 134 extracts the data stored in the readings database 146. The screening module 134 compares the extracted data from the readings database 146 to the filtered rules database 148. The screening module 134 extracts the corresponding notification stored in the rules database 148. The screening module 134 sends the extracted corresponding notification stored in the rules database 148 to the notification module 140. The screening module 134 returns to the integration module 128.

If it is determined that the screening module 134 should not be initiated then the integration module 128 determines, at step 616, if the status module 136 should be initiated. For example, if the extracted reason stored in the working database 142 was status then the integration module 128 determines to initiate the status module 136 and if the extracted reason was not status then the integration module 128 determines not to initiate the status module 136. If it is determined that the status module 136 should be initiated then the integration module 128 initiates, at step 618, the status module 136. For example, the status module 136 begins by being initiated by the integration module 128. The status module 136 filters the rules database 148 on status. The status module 136 extracts the data stored in the readings database 146. The status module 136 compares the extracted data from the readings database 146 to the filtered rules database 148. The status module 136 extracts the corresponding notification stored in the rules database 148. The status module 136 sends the extracted corresponding notification stored in the rules database 148 to the notification module 140. The status module 136 returns to the integration module 128.

If it is determined that the status module 136 should not be initiated then the integration module 128 determines, at step 620, if the evaluation module 138 should be initiated. For example, if the extracted reason stored in the working database 142 was evaluation then the integration module 128 determines to initiate the evaluation module 138 and if the extracted reason was not evaluation then the integration module 128 determines not to initiate the evaluation module 138. If it is determined that the evaluation module 138 should be initiated then the integration module 128 initiates, at step 622, the evaluation module 138. For example, the evaluation module 138 begins by being initiated by the integration module 128. The evaluation module 138 filters the rules database 148 on evaluation. The evaluation module 138 extracts the data stored in the readings database 146. The evaluation module 138 compares the extracted data from the readings database 146 to the filtered rules database 148. The evaluation module 138 extracts the corresponding notification stored in the rules database 148. The evaluation module 138 sends the extracted corresponding notification stored in the rules database 148 to the notification module 140. The evaluation module 138 returns to the integration module 128. If it is determined that the evaluation module 138 should not be initiated then the integration module 128 returns, at step 624, to the base module 120.

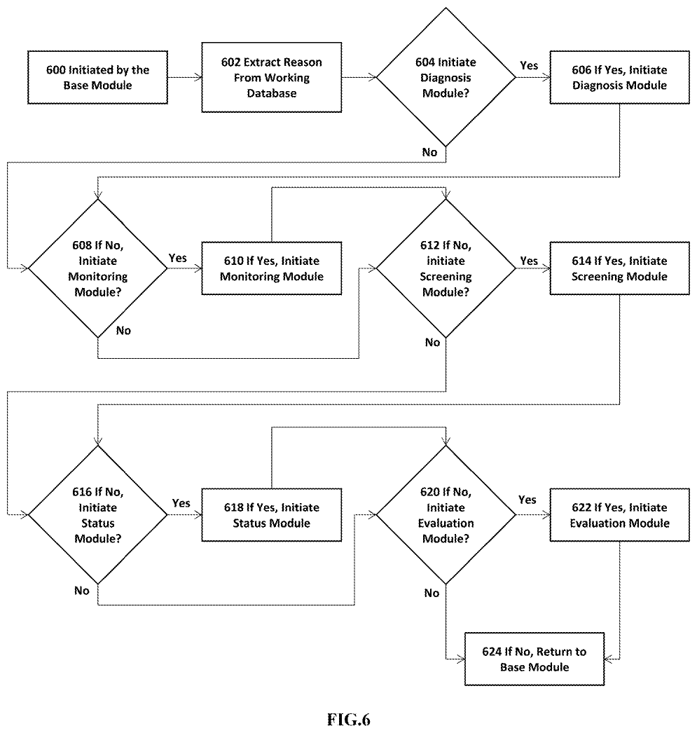

FIG. 7 illustrates a method of operation of the diagnosis module 130. The process begins with the diagnosis module 130 being initiated, at step 700, by the integration module 128. For example, the diagnosis module 130 may be initiated if the extracted reason from the working database 142 is diagnosis. The diagnosis reason may be to diagnose a disease using the collected data from the first and second analyte. The diagnosis module 130 filters, at step 702, the rules database 148 on diagnosis. For example, the diagnosis module 130 filters the rules database 148 based on the reason for diagnosis. In some embodiments, the rules database 148 may be filtered on the type of condition, such as diabetes. The diagnosis module 130 extracts, at step 704, the data stored in the readings database 146. For example, the diagnosis module 130 extracts the data stored in the readings database 146, such as the readings for the first and second analyte over a period time. For example, the extracted data may be the user's blood glucose readings from any point during the day and hemoglobin readings over a two to three month time period. The diagnosis module 130 compares, at step 706, the extracted data from the readings database 146 to the filtered rules database 148. For example, the diagnosis module 130 compares the extracted first and second analyte data from the readings database 146 to the rules database 148 to determine if the extracted data is within a range stored in the rules database 148 that has a corresponding notification. For example, if the user's blood glucose readings are between 80-130 mg/dL and hemoglobin levels are between 14-20 g/dL the corresponding notification may be the user is at risk for diabetes. For example, the two analytes measured to assist in diagnosing diabetes may be blood glucose and hemoglobin, in which blood glucose may be measured at any time during the day and hemoglobin may be a measure of person's average blood glucose levels over two to three months. For example, blood glucose may be measured by a taking the measurements when the user is fasting, such as fasting for several priors to the measurements being taken and may be taken any time during the day. In some embodiments, the blood glucose measurements may be taken at any point during the day regardless of if the user has fasted or not. The hemoglobin measurements may be a person's average blood glucose levels over two to three months, in which the measurements may be taken daily over the course of two to three months. The combination of the blood glucose being measured at any point during the day and hemoglobin being measured for two to three months may be a strong indicator if a person has diabetes. Another example may be if a person is being diagnosed with high cholesterol in which the two analytes being measured may be high-density lipoprotein cholesterol readings and low-density lipoprotein cholesterol readings. For example, if the user's high-density lipoprotein cholesterol readings are above 190 mg/dL and low-density lipoprotein cholesterol readings are lower than 40 mg/dL then the notification may be the high cholesterol treatment is working. For example, the high-density lipoprotein cholesterol readings and low-density lipoprotein cholesterol readings may be collected at any point during the day and may be collected simultaneously to determine if the person or user has high cholesterol. In some embodiments, the measurements may be collected during the day over a two to four day time period to increase the accuracy of the measurements.

The diagnosis module 130 extracts, at step 708, the corresponding notification stored in the rules database 148. For example, the diagnosis module 130 extracts the corresponding notification, such as risk of diabetes, from the rules database 148. In some embodiments, the notification for diagnosis may be not at a risk or at risk for the diagnosis and provide additional notifications or suggestions to visit a health care facility to get a full diagnosis. The diagnosis module 130 sends, at step 710, the extracted corresponding notification stored in the rules database 148 to the notification module 140. For example, the diagnosis module 130 sends the extracted notification to the notification module 140 to be displayed on the user interface 118 to inform the user. The diagnosis module 130 returns, at step 712, to the integration module 128.

FIG. 8 illustrates a method of operation of the monitoring module 132. The process begins with the monitoring module 132 being initiated, at step 800, by the integration module 128. For example, the monitoring module 132 may be initiated if the extracted reason from the working database 142 is monitor. The monitor reason may be to monitor a treatment using the collected data from the first and second analyte. The monitoring module 132 filters, at step 802, the rules database 148 on monitoring. For example, the monitor module 132 filters the rules database 148 based on the reason for monitoring. In some embodiments, the rules database 148 may be filtered on the type of condition, such as high cholesterol. The monitoring module 132 may extract the user's data from the readings database 146 and compare the data to the filtered rules database 148. For example, a person may monitor a high cholesterol treatment in which the two analytes being measured may be high-density lipoprotein cholesterol readings and low-density lipoprotein cholesterol readings. For example, if the user's high-density lipoprotein cholesterol readings are lower than 190 mg/dL and low-density lipoprotein cholesterol readings are more than 40 mg/dl then the notification may be the high cholesterol treatment is working. For example, the high-density lipoprotein cholesterol readings and low-density lipoprotein cholesterol readings may be collected for three months at any point during the day and may be collected simultaneously to determine if the person or user treatment for high cholesterol is working. In some embodiments, the measurements may be collected at the end of the treatment to determine if the high cholesterol medication was effective. In some embodiments, the data collected over the three month time period may be used to determine if the user's high-density lipoprotein cholesterol readings and low-density lipoprotein cholesterol readings improved over time. Another example may be if a person is monitoring dehydration treatments then the analytes measured may be sodium and potassium. For example, if the user's sodium readings are over 146 mEq/L and potassium levels below 3.4 mmol/L the user is in danger of dehydration. For example, the two analytes measured to assist in determining hydration may be sodium and potassium, in which when a person is dehydrated there is an increase concentration of sodium in their blood and a decrease of potassium levels in their blood. The measurements may be collected simultaneously over the course of a day every hour to determine if there condition is improving. The user may use an over-the-counter oral rehydration solution or a dilute a sports drink with water to improve their condition.

The monitoring module 132 extracts, at step 804, the data stored in the readings database 146. For example, the monitor module 132 extracts the data stored in the readings database 146, such as the readings for the first and second analyte over a period time. For example, the extracted data may be the user's high-density lipoprotein cholesterol readings and low-density lipoprotein cholesterol readings from any point during the day for three months. In some embodiments, the measurements may be collected during the day for as long as the user is prescribed the treatment in order to determine if the medication is effective. The monitoring module 132 compares, at step 806, the extracted data from the readings database 146 to the filtered rules database 148. For example, the monitor module 132 compares the extracted first and second analyte data from the readings database 146 to the rules database 148 to determine if the extracted data is within a range stored in the rules database 148 that has a corresponding notification. For example, if the user may use the device 102 to determine if a treatment or medicine for high cholesterol is working by monitoring their high-density lipoprotein cholesterol readings and low-density lipoprotein cholesterol readings over a period of time. For example, a person may monitor a high cholesterol treatment in which the two analytes being measured may be high-density lipoprotein cholesterol readings and low-density lipoprotein cholesterol readings. For example, if the user's high-density lipoprotein cholesterol readings are lower than 190 mg/dL and low-density lipoprotein cholesterol readings are more than 40 mg/dL then the notification may be the high cholesterol treatment is working. For example, the high-density lipoprotein cholesterol readings and low-density lipoprotein cholesterol readings may be collected for three months at any point during the day and may be collected simultaneously to determine if the person or user treatment for high cholesterol is working. In some embodiments, the measurements may be collected at the end of the treatment to determine if the high cholesterol medication was effective. In some embodiments, the data collected over the three month time period may be used to determine if the user's high-density lipoprotein cholesterol readings and low-density lipoprotein cholesterol readings improved over time. Another example may be if a person is monitoring dehydration treatments then the analytes measured may be sodium and potassium. For example, if the user's sodium readings are over 146 mEq/L and potassium levels below 3.4 mmol/L the user is in danger of dehydration. For example, the two analytes measured to assist in determining hydration may be sodium and potassium, in which when a person is dehydrated there is an increase concentration of sodium in their blood and a decrease of potassium levels in their blood. The measurements may be collected simultaneously over the course of a day every hour to determine if there condition is improving. The user may use an over-the-counter oral rehydration solution or a dilute a sports drink with water to improve their condition.

The monitoring module 132 extracts, at step 808, the corresponding notification stored in the rules database 148. For example, the monitor module 132 extracts the corresponding notification, such as the high cholesterol treatment is working, from the rules database 148. The monitoring module 132 sends, at step 810, the extracted corresponding notification stored in the rules database 148 to the notification module 140. For example, the monitor module 132 sends the extracted notification to the notification module 140 to be displayed on the user interface 118 to inform the user. The monitoring module 132 returns, at step 812, to the integration module 128.

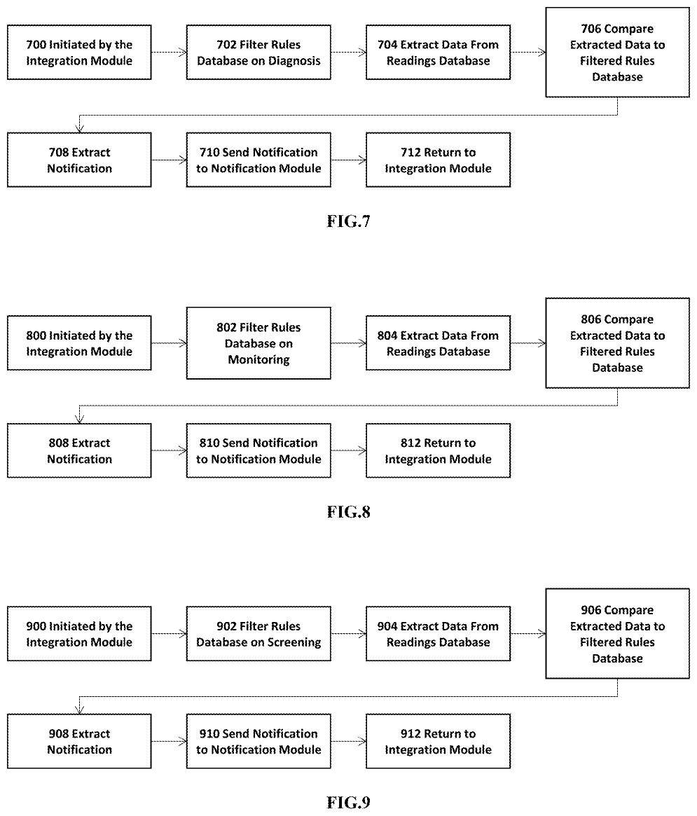

FIG. 9 illustrates a method of operation of the screening module 134. The process begins with the screening module 134 being initiated, at step 900, by the integration module 128. For example, the screening module 134 may be initiated if the extracted reason from the working database 142 is screening. The screening reason may be a screening tool used to identify individuals' risk of certain diseases using the collected data from the first and second analyte. The screening module 134 filters, at step 902, the rules database 148 on screening. For example, the screening module 134 filters the rules database 148 reason on screening. In some embodiments, the rules database 148 may be filtered on the type of condition, such as anemia. The screening module 134 may extract the data from the readings database 146, such as the blood glucose levels and hemoglobin levels and compare the extracted data to the rules database 148. For example, if the user's blood glucose readings are between 80-130 mg/dL and hemoglobin levels are lower than 13 g/dL the corresponding notification may be the user is at risk for anemia, see healthcare professional. For example, if the user has diabetes and low hemoglobin levels they may be at risk for anemia. The hemoglobin and blood glucose measurements may be taken once simultaneously to determine if the user has anemia. The blood glucose measurements may be taken once to determine if the user has diabetes and then the hemoglobin measurements may be taken over two to four weeks to determine if the user has anemia. Another example may be if a person may be screening for high cholesterol in which the two analytes being measured may be high-density lipoprotein cholesterol readings and low-density lipoprotein cholesterol readings. For example, if the user's high-density lipoprotein cholesterol readings are lower than 190 mg/dL and low-density lipoprotein cholesterol readings are more than 40 mg/dL then the notification may be the user does not have high cholesterol. For example, the high-density lipoprotein cholesterol readings and low-density lipoprotein cholesterol readings may be collected at any point during the day and simultaneously to determine if the person or user has high cholesterol.

The screening module 134 extracts, at step 904, the data stored in the readings database 146. For example, the screening module 134 extracts the data stored in the readings database 146, such as the readings for the first and second analyte over a period of time. For example, the extracted data may be the user's blood glucose readings and hemoglobin readings over a period of time. The screening module 134 compares, at step 906, the extracted data from the readings database 146 to the filtered rules database 148. For example, the screening module 134 compares the extracted first and second analyte data from the readings database 146 to the rules database 148 to determine if the extracted data is within a range stored in the rules database 148 that has a corresponding notification. For example, if the user's blood glucose readings are between 80-130 mg/dL and hemoglobin levels are lower than 13 g/dL the corresponding notification may be the user is at risk for anemia, see healthcare professional. For example, if the user has diabetes and low hemoglobin levels they may be at risk for anemia. The hemoglobin and blood glucose measurements may be taken once simultaneously to determine if the user has anemia. The blood glucose measurements may be taken once to determine if the user has diabetes and then the hemoglobin measurements may be taken over two to four weeks to determine if the user has anemia. Another example may be if a person may be screening for high cholesterol in which the two analytes being measured may be high-density lipoprotein cholesterol readings and low-density lipoprotein cholesterol readings. For example, if the user's high-density lipoprotein cholesterol readings are lower than 190 mg/dL and low-density lipoprotein cholesterol readings are more than 40 mg/dL then the notification may be the user does not have high cholesterol. For example, the high-density lipoprotein cholesterol readings and low-density lipoprotein cholesterol readings may be collected at any point during the day and simultaneously to determine if the person or user has high cholesterol.

The screening module 134 extracts, at step 908, the corresponding notification stored in the rules database 148. For example, the screening module 134 extracts the corresponding notification, such as risk of anemia, see healthcare professional from the rules database 148. In some embodiments, the notification may include corresponding symptoms for the user to be aware of to assist in determining if they should see or consult a healthcare professional, such as fatigue, weakness, shortness of breath, pale skin, dizziness, etc. The screening module 134 sends, at step 910, the extracted corresponding notification stored in the rules database 148 to the notification module 140. For example, the screening module 134 sends the extracted notification to the notification module 140 to be displayed on the user interface 118 to inform the user. The screening module 134 returns, at step 912, to the integration module 128.

FIG. 10 illustrates a method of operation of a status module 136. The process begins with the status module 136 being initiated, at step 1000, by the integration module 128. For example, the status module 136 may be initiated if the extracted reason from the working database 142 is status. The status reason may be to determine the health status of an individual using the collected data from the first and second analyte. The status module 136 filters, at step 1002, the rules database 148 on status. For example, the status module 136 filters the rules database 148 reason on status. In some embodiments, the rules database 148 may be filtered on the type of condition, such as dehydration. The status module 136 extracts, at step 1004, the data stored in the readings database 146. For example, the status module 136 extracts the data stored in the readings database 146, such as the readings for the first and second analyte over a period of time. For example, the extracted data may be the user's sodium readings and potassium readings over a period of time. The status module 136 compares, at step 1006, the extracted data from the readings database 146 to the filtered rules database 148. For example, the status module 136 compares the extracted first and second analyte data from the readings database 146 to the rules database 148 to determine if the extracted data is within a range stored in the rules database 148 that has a corresponding notification. For example, if the user's sodium readings are between 135-145 mEq/L and potassium levels are between 3.5-5.0 mmol/L the corresponding notification may be the user hydration levels are good. For example, the two analytes measured to assist in determining hydration may be sodium and potassium, in which when a person is dehydrated there is an increase concentration of sodium in their blood and a decrease of potassium levels in their blood. If the sodium and potassium levels are within what is considered a normal range then it can be determined that their hydration levels are normal or in good standing.

The status module 136 extracts, at step 1008, the corresponding notification stored in the rules database 148. For example, the status module 136 extracts the corresponding notification, such as hydration levels are good, from the rules database 148. The status module 136 sends, at step 1010, the extracted corresponding notification stored in the rules database 148 to the notification module 140. For example, the status module 136 sends the extracted notification to the notification module 140 to be displayed on the user interface 118 to inform the user. The status module 136 returns, at step 1012, to the integration module 128.

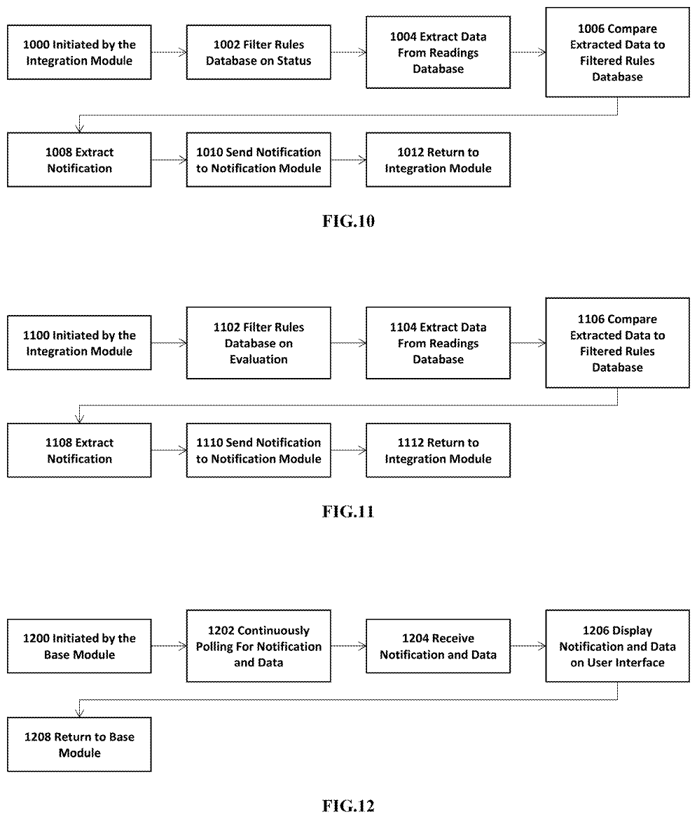

FIG. 11 illustrates a method of operation of an evaluation module 138. The process begins with the evaluation module 138 being initiated, at step 1100, by the integration module 128. For example, the evaluation module 138 may be initiated if the extracted reason from the working database 142 is evaluation. The evaluation reason may be to evaluate a person's health changes over a period of time using the collected data from the first and second analyte. The evaluation module 138 filters, at step 1102, the rules database 148 on evaluation. For example, the evaluation module 138 filters the rules database 148 reason on evaluation. In some embodiments, the rules database 148 may be filtered on the type of condition, such as dehydration. The evaluation module 138 extracts, at step 1104, the data stored in the readings database 146. For example, the evaluation module 138 extracts the data stored in the readings database 146, such as the readings for the first and second analyte over a period of time. For example, the extracted data may be the user's sodium readings and potassium readings over a period of time. The evaluation module 138 compares, at step 1106, the extracted data from the readings database 146 to the filtered rules database 148. For example, the evaluation module 138 compares the extracted first and second analyte data from the readings database 146 to the rules database 148 to determine if the extracted data is within a range stored in the rules database 148 that has a corresponding notification. For example, if the user's sodium readings are over 146 mEq/L and potassium levels below 3.4 mmol/L the corresponding notification may be the user is in danger of dehydration. For example, the two analytes measured to assist in determining hydration may be sodium and potassium, in which when a person is dehydrated there is an increase concentration of sodium in their blood and a decrease of potassium levels in their blood. If there sodium are above, and potassium levels are below what is considered a normal range then it can be determined that the user may be in danger of dehydration.

The evaluation module 138 extracts, at step 1108, the corresponding notification stored in the rules database 148. For example, the evaluation module 138 extracts the corresponding notification, such as danger of dehydration, from the rules database 148. The evaluation module 138 sends, at step 1110, the extracted corresponding notification stored in the rules database 148 to the notification module 140. For example, the evaluation module 138 sends the extracted notification to the notification module 140 to be displayed on the user interface 118 to inform the user. The evaluation module 138 returns, at step 1112, to the integration module 128.

FIG. 12 illustrates a method of operation of a notification module 140. The process begins with the notification module 140 being initiated, at step 1200, by the base module 120. For example, the notification module 40 may be initiated once the integration module 128 initiates either the diagnosis module 130, monitor module 132, screening module 134, status module 136, or evaluation module 138. The notification module 140 continuously polls, at step 1202, to receive a notification and data. For example, the notification module 140 is continuously polling to receive a notification from either the diagnosis module 130, monitor module 132, screening module 134, status module 136, or evaluation module 138 and the first and second analyte data. The notification module 140 receives, at step 1204, the notification and data. For example, the notification module 140 receives a notification from either the diagnosis module 130, monitor module 132, screening module 134, status module 136, or evaluation module 138 and the first and second analyte data. For example, the diagnosis module 130 may send the notification of risk of diabetes and the blood glucose and hemoglobin levels. For example, the monitor module 132 may send the notification of high cholesterol treatment is working and the high-density lipoprotein cholesterol and low-density lipoprotein cholesterol levels. For example, the screening module 134 may send the notification of see a healthcare professional, risk of anemia and the blood glucose and hemoglobin levels. For example, the status module 136 may send the notification of hydration levels good and the sodium and potassium levels. For example, the evaluation module 138 may send the notification of danger of dehydration and the sodium and potassium levels. The notification module 140 displays, at step 1206, the notification and data on the user interface 118. For example, the notification module 140 may display the notification, such as risk of diabetes, high cholesterol treatment is working, see healthcare professional risk of anemia, hydration levels good, danger of dehydration, etc. and the first and second analyte levels on the user interface 118. The notification module 140 returns, at step 1208, to the base module 120.

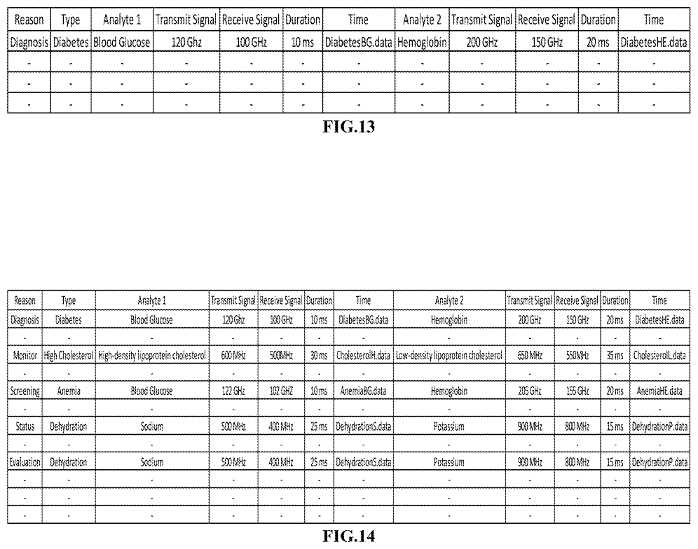

FIG. 13 illustrates an example of the working database 142. The database is created during the process described in the program module 122 and the analyte selection module 124 and contains the data used to monitor the two analytes for the user. The database contains the reason, such as diagnosis a disease, monitor a treatment, screening, monitor health status, and evaluate changes over, the type such as the type of condition the user desires to monitor such as diabetes, high cholesterol, anemia, dehydration, etc. the first analyte, the transmit signal of the first analyte, the receive signal of the first analyte, the duration the transmit signal of the first analyte should last, the time in which the transmit signal should be sent for the first analyte, the second analyte, the transmit signal of the second analyte, the receive signal of the second analyte, the duration the transmit signal of the second analyte should last, the time in which the transmit signal should be sent for the second analyte, etc. The database may be unique to the user through the user inputs collected in the program module 122 to monitor two analytes for the purpose of diagnosing a disease, monitoring a treatment, screening, monitoring health status, and evaluating changes over. The time in which the analyte data is collected may be stored in a data file, which may include a schedule to collect the data, such as every hour, daily, weekly, monthly, quarterly, yearly, etc. at certain times during the day, week, month, quarter, year, etc. and/or for a certain time period such as for a day, week, month, quarter, year, etc. In some embodiments, the detection of the analyte may be based upon comparing the transmit signals and received signals to ground truth data collected by medical devices. For example, the database may utilize a module that may be configured to execute an AI correlation between the real-time ground truth data and the RX converted data. In one embodiment, the AI correlation between the real-time ground truth data and the RX-converted data is executed to determine whether the RX-converted data corresponds to the real-time ground truth data. For example, the module may execute the AI correlation between the real-time ground truth data related to the blood glucose level of the patient as 110 mg/dl corresponding to the radio signal of frequency 122 GHZ, and the 8-bit data corresponding to Activated RF range 140-155 GHz. For example, the memory 110 may store the real-time ground truth data and RX converted data. For example, the memory 110 stores the real-time ground truth data related to the blood glucose level of the patient as 110 mg/dL corresponding to the radio signal of frequency 122 GHZ, and the 8-bit data corresponding to Activated RF range 140-155 GHZ.

FIG. 14 illustrates an example of the analyte database 144. The database contains a plurality of analytes and corresponding data to detect the analytes used by the analyte selection module 124 to extract the data corresponding to the inputs from the user. The database contains the reason, such as diagnosis a disease, monitor a treatment, screening, monitor health status, and evaluate changes over, the type such as the type of condition the user desires to monitor such as diabetes, high cholesterol, anemia, dehydration, etc. the first analyte, the transmit signal of the first analyte, the receive signal of the first analyte, the duration the transmit signal of the first analyte should last, the time in which the transmit signal should be sent for the first analyte, the second analyte, the transmit signal of the second analyte, the receive signal of the second analyte, the duration the transmit signal of the second analyte should last, the time in which the transmit signal should be sent for the second analyte, etc. The time in which the analyte data is collected may be stored in a data file, which may include a schedule to collect the data, such as every hour, daily, weekly, monthly, quarterly, yearly, etc. at certain times during the day, week, month, quarter, year, etc. and/or for a certain time period such as for a day, week, month, quarter, year, etc.

FIG. 15 illustrates an example of the readings database 146. The database contains the analyte levels collected from the transmission module 126. The database contains the date and time the analyte data was collected, the first analyte, the levels or readings of the first analyte, the second analyte, and the levels or readings of the second analyte. In some embodiments, the database may contain the RF transmit signals sent and received by the TX antenna 104 and RX antenna 106, the filters used by the RX antenna 106, the duration the transmitted signals lasted, etc. In some embodiments, the detection of the analyte may be based upon comparing the transmit signals and received signals to ground truth data collected by medical devices. For example, the database may utilize a module that may be configured to execute an AI correlation between the real-time ground truth data and the RX converted data. In one embodiment, the AI correlation between the real-time ground truth data and the RX-converted data is executed to determine whether the RX-converted data corresponds to the real-time ground truth data. For example, the module may execute the AI correlation between the real-time ground truth data related to the blood glucose level of the patient as 110 mg/dL corresponding to the radio signal of frequency 122 GHZ, and the 8-bit data corresponding to Activated RF range 140-155 GHz. For example, the memory 110 may store the real-time ground truth data and RX converted data. For example, the memory 110 stores the real-time ground truth data related to the blood glucose level of the patient as 110 mg/dL corresponding to the radio signal of frequency 122 GHZ, and the 8-bit data corresponding to Activated RF range 140-155 GHz. In some embodiments, the detection of the analyte of be performed by a module that performs convolution matching on the extracted RF transmit signal that was used by the TX antenna 104 and the received waveform from the RX antenna 106 that was converted to a digital processor readable format and the data extracted from a standard waveform database. For example, convolution is a mathematical operation on two functions, f and g, that produces a third function, f*g, that expresses how the shape of one is modified by the other. The term convolution refers to both the result function and to the process of computing it. It is defined as the integral of the product of the two functions after one is reflected about the y-axis and shifted. The choice of which function is reflected and shifted before the integral does not change the integral result. The integral is evaluated for all values of shift, producing the convolution function. The waveforms stored in memory 110 are matched with each of the individual data entries stored in the standard waveform database. First, the input signal can be decomposed into a set of impulses, each of which can be viewed as a scaled and shifted delta function. Second, the output resulting from each impulse is a scaled and shifted version of the impulse response. Third, the overall output signal can be found by adding these scaled and shifted impulse responses. In other words, if a system's impulse response is known, then we can calculate what the output will be for any possible input signal. In some embodiments, the module may also perform cross-correlation and autocorrelation and store the all the results in memory 110.

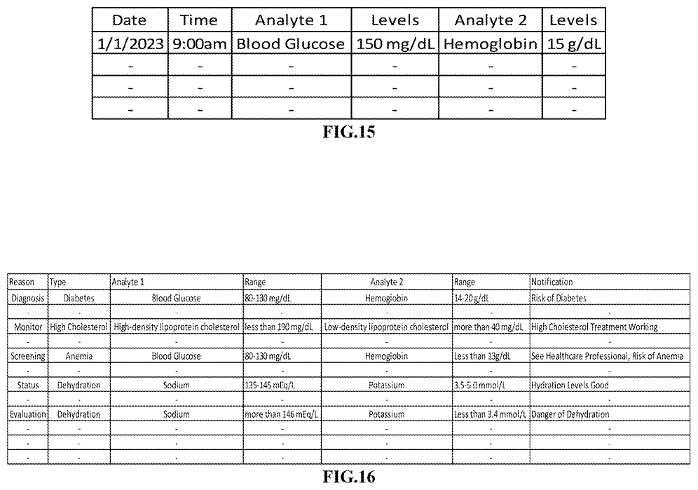

FIG. 16 illustrates an example of the rules database 148. The database is used in the process described in the diagnosis module 130, monitoring module 132, screening module 134, status module 136, and evaluation module 138 to determine if the user's analyte levels correspond with a notification that should be sent to the user. The notifications may be notifications, alerts, recommendations, suggestions, etc. to inform the user of the results of their analytes that are being monitored or tracked, such as for diagnosing a disease, monitoring a treatment, screening, monitoring health status, evaluating changes over time, etc. The database contains the reason for the user monitoring, the type of condition the user is monitoring, the first analyte, the levels or readings of the first analyte represented in a range, the second analyte, the levels or readings of the second analyte represented in a range, and the corresponding notification. For example, if the user's reason for monitoring is to diagnosis a disease and the type of condition is diabetes the first analyte may be blood glucose and the second analyte may be hemoglobin. The notification may be the user is at risk of diabetes if their blood glucose levels are between 80-130 mg/dL and hemoglobin is between 14-20 g/dL. The user's analytes may be monitored over a period of time to determine a diagnosis, for example, hemoglobin requires to be monitored for an extended period of time such as over two to three months. performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

1. A non-invasive analyte detection system, comprising:
a non-invasive radio-frequency (RF) analyte detection device that includes one or more transmit antennas configured to transmit RF analyte detection signals from the one or more transmit antennas into a user, and one or more receive antennas that receive return RF analyte signals that result from the RF analyte detection signals transmitted into the user; the non-invasive RF analyte detection device is configured to perform an analyte detection routine using the one or more transmit antennas and the one or more receive antennas;
an analog-to-digital converter connected to the one or more receive antennas;
a database in communication with the non-invasive RF analyte detection device and containing data used to control operation of the non-invasive RF analyte detection device during the analyte detection routine; the database contains a plurality of analyte monitoring data entries that differ from one another, each analyte monitoring data entry includes a reason for conducting the analyte detection routine, a type of disease condition being monitored, a first specific analyte, and a second specific analyte.

2. The non-invasive analyte detection system of claim 1, wherein each analyte monitoring data entry further includes a frequency of a first RF analyte detection signal associated with the first analyte, and a frequency of a second RF analyte detection signal associated with the second analyte.

3. The non-invasive analyte detection system of claim 2, wherein each analyte monitoring data entry further includes a frequency of a return RF analyte signal associated with the first RF analyte detection signal and a frequency of a return RF analyte signal associated with the second RF analyte detection signal.

4. The non-invasive analyte detection system of claim 2, wherein each analyte monitoring data entry further includes a time at which the first RF analyte detection signal is to be sent, and a time at which the second RF analyte detection signal is to be sent.

5. The non-invasive analyte detection system of claim 2, wherein each analyte monitoring data entry further includes a duration that the first RF analyte detection signal should last, and a duration that the second RF analyte detection signal should last.

6. The non-invasive analyte detection system of claim 2, further comprising:
a readings database in communication with the non-invasive RF analyte detection device and containing data regarding the first specific analyte and the second specific analyte obtained by the non-invasive RF analyte detection device during the analyte detection routine;
a rules database that contains data that is compared with the data in the readings database, the rules database contains a plurality of rule data entries that differ from one another; each rule data entry includes the reason for conducting the analyte detection routine, the type of disease condition being monitored, the first specific analyte, a range for the first specific analyte, the second specific analyte, and a range for the second specific analyte.

7. The non-invasive analyte detection system of claim 6, wherein each rule data entry further includes a notification.

8. An analyte monitoring method, comprising:
receiving electronic inputs from a user that include an entered reason for conducting the analyte monitoring and an entered type of disease condition to be monitored;
based on the received electronic inputs, obtaining electronic analyte monitoring data from an electronic database that contains a plurality of analyte monitoring data entries that differ from one another, each analyte monitoring data entry includes a reason for conducting the analyte monitoring, a type of disease condition, a first specific analyte, and a second specific analyte;
the electronic database is in communication with a non-invasive radio-frequency (RF) analyte detection device so that the obtained electronic analyte monitoring data is able to control the non-invasive radio-frequency (RF) analyte detection device to perform an analyte detection routine.

9. The analyte monitoring method of claim 8, further comprising using the obtained electronic analyte monitoring data to control the non-invasive RF analyte detection device to perform the analyte detection routine.

10. The analyte monitoring method of claim 8, wherein each analyte monitoring data entry further includes a frequency of a first RF analyte detection signal associated with the first analyte, and a frequency of a second RF analyte detection signal associated with the second analyte.

11. The analyte monitoring method of claim 10, wherein each analyte monitoring data entry further includes a frequency of a return RF analyte signal associated with the first RF analyte detection signal and a frequency of a return RF analyte signal associated with the second RF analyte detection signal.

12. The analyte monitoring method of claim 10, wherein each analyte monitoring data entry further includes a time at which the first RF analyte detection signal is to be sent, and a time at which the second RF analyte detection signal is to be sent.

13. The analyte monitoring method of claim 10, wherein each analyte monitoring data entry further includes a duration that the first RF analyte detection signal should last, and a duration that the second RF analyte detection signal should last.

14. The analyte monitoring method of claim 9, further comprising:
storing data obtained by the non-invasive RF analyte detection device during the analyte detection routine in a readings database;
comparing data stored in the readings database with data from a rules database, the rules database contains a plurality of rule data entries that differ from one another; each rule data entry includes the reason for conducting the analyte monitoring, the type of disease condition, the first specific analyte, a range for the first specific analyte, the second specific analyte, and a range for the second specific analyte.

15. The analyte monitoring method of claim 14, wherein each rule data entry further includes a notification.
